(19) United States
(12) Patent Application Publication
(10) Pub. No.: US 2016/0331649 A1
(43) Pub. Date: Nov. 17, 2016

(54) SKIN TREATMENT METHOD

(71) Applicant: TRANSFER DEVICES INC., Fremont, CA (US)

(72) Inventor: Charles D. Schaper, Union City, CA (US)

(73) Assignee: TRANSFER DEVICES INC., Fremont, CA (US)

(21) Appl. No.: 14/792,832

(22) Filed: Jul. 7, 2015

Related U.S. Application Data
(60) Provisional application No. 62/162,177, filed on May 15, 2015.

(51) Int. Cl. A61K 8/02; A61K 8/81

(52) U.S. Cl. CPC A61K 8/0212; A61K 8/8129; A61K 2800/884

(57) ABSTRACT

A dissolvable polymer thin film of polyvinyl alcohol is manufactured and placed in contact with the skin, or through a moisturizing layer by a cream, and lightly sprayed on the exposed side with water to achieve conformal contact with the skin by transition from a solid state, to a partial liquid/solid state, and then back to solid state upon drying. After the polymer thin film dries, which undergoes a tightening effect on the surface of the skin, a number of advantages are achieved, including a smoothed appearance, firmness, prevention of loss due to evaporation of the intervening cream thereby improving residence time of the hydrating agents, deeper diffusion of hydrating elements into the skin through a reduced volume.

### CROSS REFERENCE TO RELATED APPLICATION

This application claims priority from provisional application No. 62/162,177 filed May 15, 2015 entitled Conformal Polymer Thin Film, Soluble and Detachable, for Skin Care.

### TECHNICAL FIELD

This invention relates to the field of skin care and, in particular, to a method of cosmetic treatment of skin by application of a material layer.

### BACKGROUND ART

For personal care, there are a number of creams and ointments on the market that enhances the appearance and moisturizes the skin, which contain a wide variety of ingredients coordinated in a carrier liquid, which can be based in solvents that include water and alcohols. To achieve contact uniformly with the skin, the use of a liquid allows the material to be placed across the undulating surfaces of the body including the hands, torso, arms, and face, as well as the associated micro-crevices and skin irregularities including hair.

A disadvantage of using cream to cover the skin is that the liquid which is required as the volatile carrier dries quickly to the environment. Consequently, the materials of interest have minimal time to interact by liquid diffusion with the surrounding cellular environment, and achieve intercellular transport to achieve the desired effect. If the liquid is non-volatile, it maintains an oily appearance that is difficult to clean-up and has a glossy look that may not be appealing, and may not enable the materials to achieve intercellular transport because of its hydrophobic nature.

In addition, creams exhibit little tension, whether wet or after drying, and therefore have difficulty achieving a significant firming effect through molecular interactions across the surface of the skin.

Moreover, even though the creams are applied, some of it does not come into contact with the skin, and therefore is of no use, other than to set up a concentration gradient to enhance diffusion. But, that gradient may be established over a large dimensional area which does not enhance the gradient in an optimal fashion as the boundary layer may be significant.

U.S. Published Application 2010/0055161 describes a skin care device, such as a face mask, made of a biocompatible hydrogel layer and a backing layer. Any ingredient in a skin care composition can be formulated into the hydrogel layer of the skin care device, including PVA. The treating hydrogel layer can include any of a long list of therapeutic agents, medicines and chemicals and almost any material that can form a gel.

U.S. Pat. No. 5,939,093 describes a sheet-type cosmetic pack, wherein a cosmetic composition layer comprising a cation-based water-soluble copolymer. The sheet-type cosmetic pack of this invention is stated to have the following properties a) the keratotic plugs can be effectively removed from the skin, b) maintenance of clean skin pores imparts flexibility to the skin of a nose, c) a shortened drying time prevents a residue of cosmetic substances in the skin after a peel-off, and d) pain is mitigated due to easy peel-off.

Published PCT application WO 2001024770 describes a skin cleansing agent in the form of a preparation which can flow or can be spread on the skin and is capable of forming a film which can be peeled off the skin after drying and which contains dissolved film-forming and adhesive polymers dissolved in an aqueous-alcoholic carrier, whereby the film-forming polymer is a partially acetylated polyvinyl alcohol with 10-40 Mol % vinyl acetate-groups and the adhesive polymer is a polyvinyl-pyrrolidon. The film can be peeled off the skin after a short drying time of less than 15 minutes as an integral film. Dirt, fat, dead skin cells and black heads can thereby be removed. A preferred layer material is an ionic polymerisate of 0.1 to 10 weight % containing ethanol or isopropanol or a mixture of ethanol and isopropanol in an amount of 5 to 30 weight %.

PVA is a polymer film that is manufactured and sold commercially in sheet form as Pellon Sol-U-Film, a material that is used as a backing in embroidery.

In the semiconductor industry, spin coating apparatus is known for making thin flat films on planar surfaces, such as wafers or glass photomasks. In U.S. Pat. No. 5,234,499, Sasaki et al. describe a spin coating apparatus for use in applying a coating solution in film form to an upper surface of a substrate. This apparatus includes a rotary table for spinning the substrate as supported thereon in horizontal posture, the rotary table having a size larger than an outline contour of the substrate, and an upper rotary plate disposed parallel to and slightly spaced from the upper surface of the substrate supported on the rotary table. The rotary table and upper rotary plate define a flat treating space. A nozzle plate is disposed between the rotary table and the substrate supported thereon. The nozzle plate receives cleaning liquid supplied through a rotary shaft of the rotary table, and directs the liquid to the lower surface of the substrate. PVA has been manufactured in the semiconductor industry for use with wafers and photomasks.

Although face masks are known for treatment of skin conditions, a key aspect of a mask is intimate contact with pores. Microscopic bumps in masks defeat attempts to bring treating agents directly into contact with skin which itself is a bumpy surface. An object of the invention was to devise a mask application method for bringing skin treating agents into intimate contact with the skin of the face as well as the body.

### BRIEF SUMMARY OF THE INVENTION

The above object has been achieved with the discovery of a nanostructured mask that is made from a spin cast film, preferably of polyvinyl alcohol (PVA), for use in bringing skin treating agents into intimate contact with skin. The film is made by spin casting onto a substrate and is removed from a spinner mechanism, heated to remove water solvents, then placed on the surface of the skin. The skin is pre-treated, such as with cream, and then an applicator, such as a spray bottle, applies water to the PVA film to partially dissolve the PVA film, and thereby inducing conformal contact with the skin, or cream on the skin. The PVA film is left on the face to dry, and then as desired, is removed by peeling off the skin, or by washing away with water.

The spin coating mechanism has a flat glass table with etched, closely spaced, nanostructures, e.g. random lines spaced apart by nanometers, to create some sub-microscopic roughening, much smaller than the bumps that occur in common PVA films. The roughened film will push the pre-treatment fluid material into pores.

Besides spin coating, the key element is to achieve a thin filth of PVA liquid material on the etched glass table, prior to the drying process. Other methods that would thereby be applicable include slit coating and similar techniques.

The PVA film then over time keeps the cream moist because it prevents the rapid evaporation of the solvent in the cream. Moreover, the properties of the film prevent significant mixing of the cream into the PVA matrix, thereby keeping the two disassociated. When peeled away, the cream still maintains connection with the surface of the skin.

In addition, the PVA film, through its constriction during the drying process, induces a smaller volume under which the cream occupies, thereby inducing a greater concentration gradient at the skin surface, and promoting more in depth diffusion into the skin layers, and the irregularities of the skin.

The PVA film as it dries contracts, and therefore applies moderate tension across the surface of the skin, leading to an association of firmness and smoothness, which can be observed visually. Even after removed, the skin still feels a tightness, firmness, and youthfulness, as well as provides feedback to the user that the product is functioning properly.

When the PVA film is in place, it has a glossy and youthful appearance to the skin, thereby providing feedback to the user of its effectiveness.

The PVA film even after removed, induces a filling mechanism and smoothing and glossy appearance of the skin, because of the introduction of the cream into the skin crevices.

The PVA film can be applied with nanostructures and microstructures in the PVA film for textural and visual impact.

The PVA film can be coated with various materials of need, such as vitamins and ointments, and transfer of these materials to the skin.

The PVA film is biodegradable and as it is water soluble, is easy to clean-up, and is a safe material to utilize on the skin without allergic reaction.

### BRIEF DESCRIPTION OF THE DRAWINGS

FIG. 1 is a spinner mechanism used to make PVA film in accordance with the invention.

FIG. 2 is a top plan view of a PVA film with magnified nanostructures.

FIG. 3 is 4 plan view of application of nanostructured PVA film to skin.

FIGS. 4-6 are plan views of sequential application of PVA nanostructured film to a lotion or moisturizer layer, followed by solid-to-semi liquid phase change of the film, followed by drying and shrinking of the film.

FIG. 7 is a plan view of a PVA film applied directly to skin.

FIG. 8 is a plan view of PVA squares applied to a human face.

FIG. 9 is a plan view of a PVA sheet applied to a human face.

### DETAILED DESCRIPTION OF THE INVENTION

With reference to FIG. 1, housing 10 has an exemplary spin chuck 11 in a spin coating apparatus of the type used in the semiconductor industry for coating wafers and photomasks. A water soluble PVA solution is spread over a glass substrate with nano structures by centrifugal force to form a thin film. The PVA film needs to be of a thickness and water content that permits rapid dissolving when subjected to a water mist. The PVA film thickness should be optimized to achieve sufficient shrinking or stretching after drying on top of a cream or cosmetic applied to the skin. This process is stimulates sensory perception by a user, serving as feedback of product progression, as well as providing a smooth appearance and constricting the volume of the cream or other underlying material.

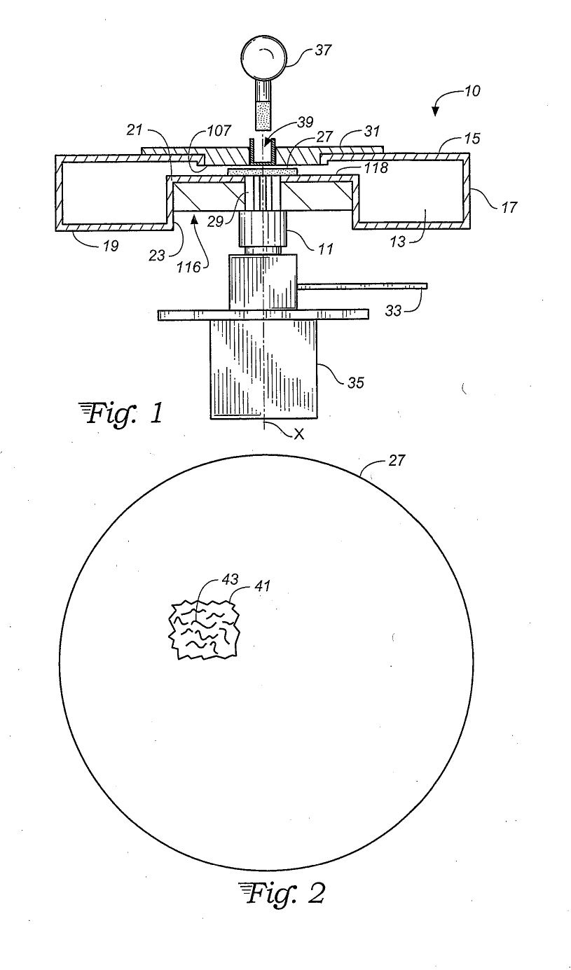

The PVA coating solution must be applied in an excessive amount with the excess captured at the outer periphery of the substrate. The nano structures on the substrate are random lines with nanometer spacing described below with reference to FIG. 2. Returning to FIG. 1, a toroidal chamber 13 has a top wall 15, an outer sidewall 17 extending downwardly from top wall 15, a bottom wall 19 and internal walls including an upper intermediate wall 21 and a side intermediate wall 23. Chamber 13 is used to capture excess coating solution and to provide a large air receiving zone that prevents air backflow onto the substrate by forming eddies. The top wall 15 includes an opening for substrate 27.

The upper wall 21 includes a plurality of vacuum holes 29 extending therethrough. A lid 31 is removable to reveal an opening 33 so that substrate 27 may be placed on the upper wall surface to cover vacuum holes 29. Vacuum pressure holds the substrate in place. Opening 33 is used for inserting a dispenser for PVA solution. A PVA solution dispenser 35 dispenses coating solution within the opening 33.

In operation, a glass substrate 27 having nano structures is inserted into the housing and placed on the upper wall 21. The substrate may be positioned on wall 21 using tweezers or a robotic handler. The substrate 27 is secured in the housing on the spin chuck using vacuum pressure via vacuum source 33. When lid 31 is closed, a motor 35 is operable to cause the spin chuck 11 and substrate 27 to rotate about the spin chuck axis at a typical rotation rate of, for example, 600 rpm for a brief period of time, for example ten seconds. Duration of spinning will control thickness of the coating.

The lowermost portion of the lid is many millimeters above glass substrate 27 to permit drying of the material. The substrate is centered on the spin axis by an alignment device. The housing 10 and spin chuck 11 rotate coaxially. As the spin chuck 11 and attached housing holding substrate 27 are rotating, or prior to rotation, a coating solution, such as PVA, is dispensed with dispenser 37. Dispensing is through the opening 39 such that the substrate 27 is coated with coating solution by centrifugal force of spin chuck rotation. Excess coating material flows off of the substrate and is collected in chamber 13 for recycling. After coating, the chamber may be heated for partial air drying of the film. After a partial drying process in ambient air, the PVA film and substrate are placed in an oven for complete drying and sterilization purposes. The film is then removed, subjected to UV light, again for sterilization purposes, and then cut into its final size upon peeling the PVA film away from the glass plate.

In FIG. 2, the substrate 27 has a magnified region 41 with nanostructures 43. The nanostructures 43 are random lines separated from each other by a few nanometers. The impressions from nanostructures on the substrate are transferred to corresponding structures on the thin-film. After peeling the thin film from the substrate, the film may be cut into squares or rectangles to eliminate any edge tears or removal marks. The nanostructures on the etched glass surface may range from several 1000 nm down to near perfect planarity of sub-10 nm.

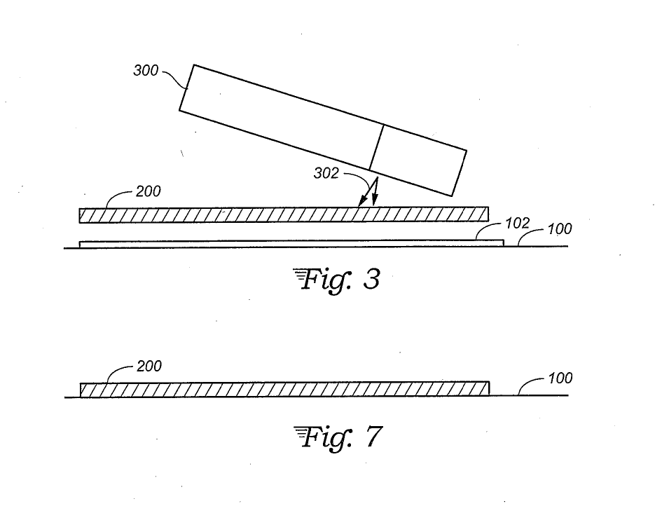

In use and with reference to FIG. 3, a water soluble nanostructured film, made of PVA, 200, is presented to the surface of the skin 100, which can be pre-moistened with moisturizer 102 or other skin treatment material described above, such as creams or lotions or serums. In this application all moisture layers are referred to as creams but the term “creams” is intended to encompass lotions, moisturizers and miscellaneous skin treatment materials. Similarly, the term “creams” is used interchangeably with lotions and moisturizers. The moisturizer may be conditioned with a mist of water prior to presentation of the PVA film. After contact of the PVA nanostructured film to the cream, a water mist 302 is sprayed on the PVA film from an applicator bottle 300.

After the applicator is used to lightly apply water to the external surface of the PVA nanostructured film, the PVA film achieves conformal contact to the skin by undergoing a solid to partial liquid/solid transformation. PVA nanostructured film, in the drying process, undergoes constriction to achieve tight conformal contact to the undulations induced by the skin surface and pores. Moreover, the constriction will induce a higher concentration gradient of the agents of the moisturizer, lotion or cream, thereby achieving deeper diffusion into the skin layers, because of the reduction of volume. In addition, the sealing mechanism of the PVA film through conformal encapsulation will prevent the evaporation of solvents to the environment. In addition, in facial areas that are open, such as the mouth, eyes and nostrils, the PVA film will become separated or recede to the periphery, which aids in ease of use.

After the PVA nanostructured film is then allowed to dry, which can take approximately 5-15 minutes depending on the amount of water mist applied, and then after a period of time, for example 30-45 minutes, the dried PVA film is physically removed from the skin, as shown in FIG. 3, by simple peeling the PVA film from the skin in holding an edge of the PVA film and detaching. If desired, the PVA nanostructured film can also be removed by a water rinse. The result after removing is a smooth appearance and refreshed feeling, with improved appearance of fine line removal and firmness due to deep hydrating element.

After a period of time, for example less than a day, it is possible to regenerate the near instantaneous association of firmness of the skin by applying water to the face which will activate the solid-to-liquid-to-solid transition of any remaining PVA molecules, which will improve the firmness because of the constriction of the PVA molecule.

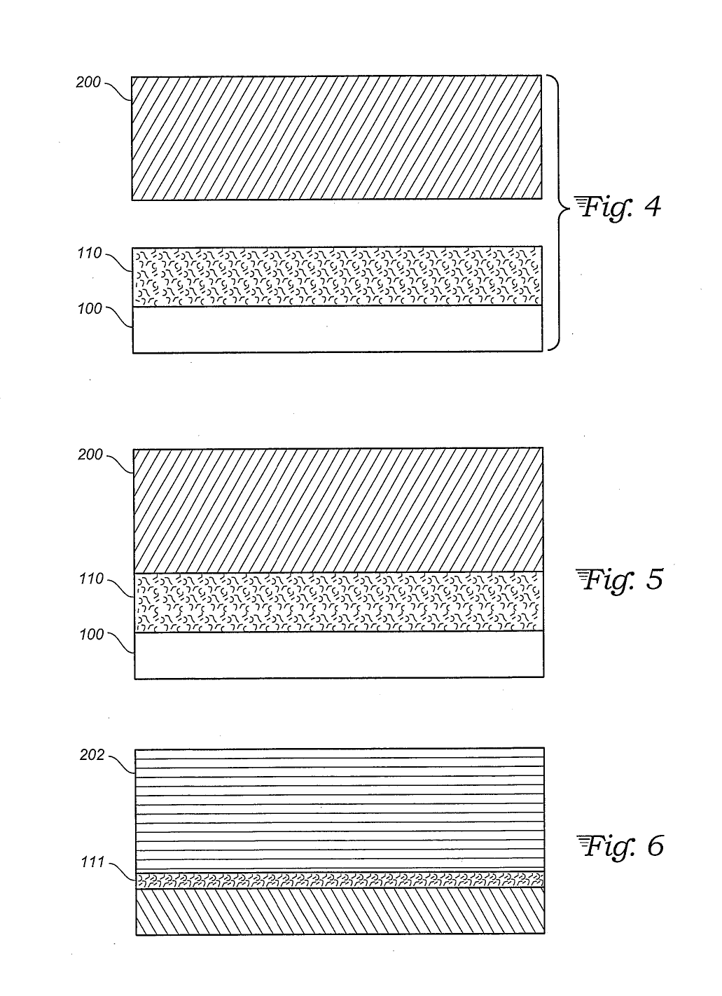

In FIG. 4, the process of placing the PVA nanostructured film 200 on the moisturizer or lotion 110 is seen, with the moisturizer in intimate contact with skin layer 100. In FIG. 5, application of liquid to the film by subjecting the PVA nanostructured film to water mist causes to film to undergo conforming and a solid to liquid transition, followed by a return to solid upon drying. At first, the film softens or relaxes upon contact by water mist and loses its two-dimensional areawise character, assuming instead a three-dimensional character, because it is not supported by an integral backing layer. This relaxation of the film causes the film to slump onto the lotion layer on the face and conform to the face, with the film at least partially dissolving into the lotion. Conforming to the face is greater than if a similar dry sheet is pressed against the face. Drying is by liquid evaporation since the sprayed liquid is not chemically bound and is sprayed in an amount that is in excess of. This is followed by a volume constriction effect, shown in FIG. 6, wherein the cream, lotion or moisturizer achieves a deeper penetration into the skin and a prevention of evaporation of the solvents of the cream, and a smoother profile of the cream to the smooth PVA nanostructured film. The concentration gradient of material within the film increases because of the smaller volume to permit deeper penetration. The facilitated action of pushing the material into the skin, i.e. application of force, is achieved as well, and the prevention of evaporation of solvents within the cream is achieved by encapsulation, which helps to hydrate the skin layers through penetration.

FIG. 7 shows applying and processing the PVA nanostructured film directly onto the skin, without an intervening lotion, which will achieve intimate conformal contact. While the benefits of the lotion to hydrate and smooth the skin are not apparent, the firmness will remain because of the nature of the PVA material after removal. In addition, the result will achieve a deep cleansing of the skin because of the tight conformal contact of the skin to the face. Consequently, it will be slightly more resistant to peel away the PVA nanostructured film, and therefore a water wash may be preferred to remove it.

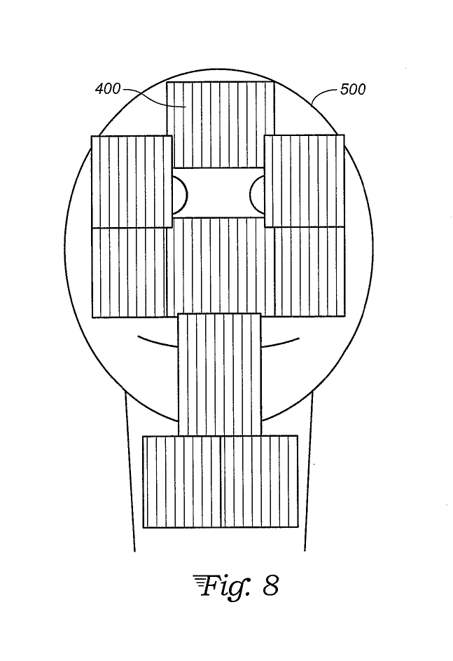

To apply the PVA nanostructured films to the facial and/or neck area, a distribution of smaller sized PVA films can be utilized, such as 3.5″×3.5″ squares, and placed on the surface of the skin, and processed individually. A cream is first applied to the skin, then a water mist is sprayed on the surface of the cream, and then a thin PVA nanostructured film sheet is applied to the area 400 of the face 500 in FIG. 8. A water mist is then applied to the film so that the film is exposed to water mist from both sides. The slumping film then achieves conformal contact with the cream, thereby encapsulating the cream or lotion against the skin. It is recommended that two such square films be applied to the forehead area, half-sheets of square films to the temples, one film each to the cheeks, one film to the nose area, one film to the mouth area, and three films spread across the neck, for a total of ten films. After drying, the formed film can be removed from the face and neck as a single entity.

Over areas such as the eyes, nostrils, or mouth, which are open, the film detaches from this region because it has nothing to bond onto, and therefore is either removed or pushed to adjacent skin areas. This process is repeated as desired over the entire region. A period of time is elapsed to dry the films, for example 15 minutes, or longer if desired and then peeled away from the skin.

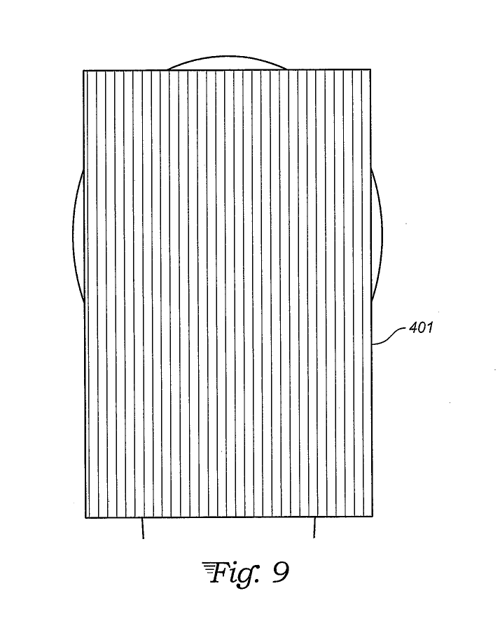

To improve the ease of application from multiple sheets to a single sheet, a large PVA nanostructured film 401 can be placed over the targeted regions of the skin, as in FIG. 9, and then applied with a water mist to achieve conformal contact to the skin. The open areas of the face, because of the thin film nature of the PVA nanostructured material design, will fall off to the side, and therefore not prevent the opening of the areas, improving safety.

In addition to the face and neck areas, the nanostructured PVA films can be applied to other areas of the body where smoothness may be desired, as well as the constricting effect during the drying period of the PVA film. For example, other areas include the torso, arms, legs, as well as the hands and feet. The procedure is the same as that described of the facial and neck areas, in which the moisturizing cream is placed on the skin, subjected to a water mist, placed with the nanostructured PVA film, and then subjected to further water mist, and drying to achieve conformal contact. Removal is performed by peeling or by water rinse.

To promote the effect in drying, it is possible to include aids, such as forced convective fans, or heating lamps. The use of a heating lamp will promote the effective drying and volume constriction, resulting in tight conformal fit of the nanostructured PVA film on the surface of the skin.

The use of the technology is described for the completely water-soluble film in a size approximately 3.25″×3.25″ called an Overlay, and is applied as follows: (A) First apply Cream to the facial and neck areas. (B) Apply 10 Overlays to the full face and neck as follows: 2 on the forehead; ½ to each temple; 1 to each cheek; 1 to the nose; 1 to the mouth area; 3 on the neck. Start with the forehead and work downward. Begin by spraying the Water Mist over the right half of the forehead, and then place one Overlay at the top of the forehead at the hairline. Bring the Overlay down to the top of the eyebrow, and fold the overhanging portion upwards. Repeat the process with the left side of the forehead. To move onto the temples, tear one Overlay in half, then apply to each side of the face along the temple. Afterwards, place one Overlay on each side of the face on the cheek area, extending from the hairline to the nasal area. To cover the nose, place an Overlay over it, folding it as necessary to achieve coverage. Use the process of spraying Water Mist over the mouth region, place an Overlay on the mouth and chin area, then spray Water Mist again, with the mouth region will separate due to dissolution from the water. Complete the process for the neck, using one Overlay in the center of the neck, and an Overlay to each on the left and right side of the neck. (C) Allow sufficient time for complete drying, which will take approximately 15 to 20 minutes, depending upon the amount of water applied. At this point, the dried Overlays can be removed, but if a longer effective period is desired, it can be removed after 1-2 hours, or longer. The removal of the Overlays can be peeling off the dried film, by a water rinse, or by using a moistened towel. After use, a general water rinse can be applied. The experience of the overlays can be improved by doubling the Overlays at desired areas, for example, the temple. In addition, a fan can be used to promote rapid drying and increase the tightening effect of the Overlay when it transitions to a dried state.

1. A skin treatment method comprising:
placing a layer of cream on human skin,
placing a completely water soluble film on the cream layer, and
spraying the water soluble film with water until softening occurs.

2. The method of claim 1 wherein the water soluble film is comprised of a PVA film.

3. The method of claim 2 wherein said PVA film is nanostructured.

4. The method of claim 1 wherein said spraying the water soluble film with water dissolves the film onto the cream.

5. The method of claim 1 further defined by drying the water soluble film by water evaporation thereby solidifying the film.

6. The method of claim 5 further defined by peeling the dried film from the cream.

7. The method of claim 5 further defined by rinsing off with water the dried film from the cream.

8. The method of claim 5 further defined by using a damp towel to remove the dried film from the cream.

9. The method of claim 5 further defined by drying the film until constriction occurs pushing the water soluble film toward the skin.

10. The method of claim 9 wherein the water soluble film is applied to the cream over a surface encapsulating the cream.

11. The method of claim 1 further defined by spraying water on the cream layer before placing the water soluble film on the cream layer.

12. The method of claim 1 further defined by creating nanostructures on the water soluble film by creating corresponding nanostructures on a substrate used to form the film.

13. The method of claim 1 further defined by drying the water soluble film on the skin by forced convection.

14. The method of claim 1 further defined by drying the water soluble film with radiative heat lights.

15. The method of claim 3 wherein the nanostructured film is microstructured.

16. The method of claim 3 wherein the nanostructured film is planar.

17. The method of claim 1 wherein the cream comprises a moisturizer, fade cream, serum, or lotion.

18. The method of claim 1 wherein the skin can be associated with the face, neck, torso, arm, legs, hands or feet.

19. The method of claim 1 wherein the water-soluble film is coated with an oil-based material prior to contact with the cream.

20. The method of claim 1 wherein the water soluble film is formed by spin casting the film.

21. The method of claim 20 wherein the spin casting of the film is by spinning a nanostructured substrate about an axis and applying a water soluble layer to the nanostructured substrate, drying the water soluble layer and peeling the water soluble layer from the substrate.

22. The method of claim 1 wherein the water soluble film is formed slit coating the film.

23. A facial treatment method comprising:
placing a nanostructured PVA film on a human face,
spraying the PVA film with water until softening occurs,
drying the film by water evaporation thereby solidifying the film, and
peeling the dried film from the face.

24. The method of claim 23 wherein the placing of film on a human face is by placing a plurality of films, each film covering a portion of a face.

25. The method of claim 23 wherein the film being placed has an opening to allow for the placement of the film over a specific portion of the face.

26. The method of claim 23 comprising applying a layer of cream to the face before placing a nanostructured PVA film to the cream layer on a human face.

27. The method of claim 23 further defined by creating nanostructures on the film by creating corresponding nanostructures on a substrate used to form the film.

28. The method of claim 23 wherein the nanostructured PVA film is formed by spin casting the film.

29. The method of claim 27 wherein the spin casting of the film is by spinning a nanostructured substrate about an axis and applying a PVA layer to the nanostructured substrate, drying the PVA layer and peeling the PVA layer from the substrate.

30. The method of claim 27 wherein the nanostructures are random lines spaced from each other by nanometers.

31. The method of claim 23 wherein the nanostructured PVA is microstructured.

32. The method of claim 23 wherein the nanostructured PVA is planar.

33. The method of claim 27 wherein the substrate is a glass substrate.

34. The method of claim 26 further defined by spraying water on the cream layer before placing the nanostructured PVA film on the cream layer.

35. The method of claim 23 wherein the spraying with water is by spraying a mist.

36. The method of claim 23 wherein the nanostructured PVA film is applied to the skin on the torso, arms, legs, feet or hands.

37. The method of claim 23 wherein the drying of the nanostructured film induces a constriction of the film.

38. The method of claim 23 wherein the removal of the dried film is performed by a water rinse to dissolve the dried film and wash off.

39. The method of claim 23 wherein the removal of the dried film is performed by using a water moistened towel to dissolve the dried film and remove the film, by the fibers of the towel.

40. The method of claim 23 where the facial treatment includes the neck area from the shoulders to the jaw line.
